(19) United States
(12) Patent Application Publication
(10) Pub. No.: US 2020/0325436 A1
(43) Pub. Date: Oct. 15, 2020

(54) Systems and Methods For Isolating Microvessels From Adipose Tissue

(71) Applicant: Advanced Solutions Life Sciences, LLC, Louisville, KY (US)

(72) Inventors: James Hoying, Manchester, NH (US); Hannah Strobel, Manchester, NH (US); Sarah Bushman, Manchester, NH (US)

(73) Assignee: Advanced Solutions Life Sciences, LLC, Louisville, KY (US)

(21) Appl. No.: 16/843,951

(22) Filed: Apr. 9, 2020

Related U.S. Application Data
(60) Provisional application No. 62/831,765, filed on Apr. 10, 2019.

(51) Int. Cl. C12M 1/00

(52) U.S. Cl. CPC C12M 45/09; C12N 5/0068; C12M 45/05

(57) ABSTRACT

Methods and systems to isolate microvessels using an enriched or purified enzyme to dissociate tissue are described. The systems and methods include a second digestion to digest a top layer, from a first digestion and first centrifuge operation, with the enriched or purified enzyme to generate a second fat-enzyme solution, a second centrifuge operation, and isolation of the microvessels from pellets generated by the first and second centrifuge operations. The systems and methods may include washing the second fat-enzyme solution with an enzyme inhibitor in a post-digestion wash.

### CROSS-REFERENCE TO RELATED APPLICATIONS

The present disclosure claims the benefit of U.S. Provisional App. No. 62/831,765, filed Apr. 10, 2019, entitled “SYSTEMS AND METHODS FOR ISOLATING MICROVESSELS FROM ADIPOSE TISSUE,” the entirety of which is incorporated by reference herein.

### TECHNICAL FIELD

The present disclosure relates to a method for isolating microvessels from adipose tissue, and, more specifically, a method for isolating microvessels from adipose tissue utilizing an enriched or purified enzyme.

### BACKGROUND

A practiced method of isolation of cells, including microvessels, from tissues utilizes crude preparations of tissue disassociated enzymes to disassociate the tissue into respective cellular constituents. A need exists for alternative high-quality, efficient, and effective methods to dissociate tissue and isolate microvessels.

### BRIEF SUMMARY

According to the subject matter of the present disclosure, methods and systems may isolate microvessels using an enriched or purified enzymes to dissociate tissue. The systems and methods may include a double digestion feature and/or an immediate post-digestion wash feature.

According to an embodiment, a system to isolate microvessels using enriched enzymes to dissociate tissue may include one or more processors, a non-transitory memory communicatively coupled to the one or more processors, and machine readable instructions stored in the non-transitory memory. The enriched enzyme may include an enriched or purified enzyme. The machine readable instructions may cause the system to perform at least the following, as one or more protocols, when executed by the one or more processors: digest, in a first digestion, a minced adipose with an enriched enzyme to generate a first fat-enzyme solution, centrifuge the first fat-enzyme solution from the first digestion in a first centrifuge operation to generate one or more first pellets and a top fat layer disposed above the one or more first pellets, digest, in a second digestion, the top fat layer with the enriched enzyme to generate a second fat-enzyme solution, centrifuge the second fat-enzyme solution from the second digestion in a second centrifuge operation to generate one or more second pellets, and pass one or more portions of the one or more first pellets and the one or more second pellets through one or more screens to generate a plurality of isolated microvessels.

According to another embodiment, a method to isolate microvessels using enriched enzymes to dissociate tissue may include digesting, in a first digestion, a minced adipose with an enriched enzyme to generate a first fat-enzyme solution, centrifuging the first fat-enzyme solution from the first digestion in a first centrifuge operation to generate one or more first pellets and a top fat layer disposed above the one or more first pellets, and digesting, in a second digestion, the top fat layer with the enriched enzyme to generate a second fat-enzyme solution. The method may further include centrifuging the second fat-enzyme solution from the second digestion in a second centrifuge operation to generate one or more second pellets, and passing one or more portions of the one or more first pellets and the one or more second pellets through one or more screens to generate a plurality of isolated microvessels.

According to yet another embodiment, a method to isolate microvessels using enriched enzymes to dissociate tissue may include digesting, in a first digestion, a minced adipose with an enriched enzyme to generate a first fat-enzyme solution, using an additional enzyme as a catalyst for digestion of the first fat-enzyme solution, centrifuging the first fat-enzyme solution from the first digestion in a first centrifuge operation to generate one or more first pellets and a top fat layer disposed above the one or more first pellets, and digesting, in a second digestion, the top fat layer with the enriched enzyme to generate a second fat-enzyme solution. The method may further include washing the second fat-enzyme solution with an enzyme inhibitor in a post-digestion wash, centrifuging the second fat-enzyme solution from the second digestion in a second centrifuge operation to generate one or more second pellets, and passing one or more portions of the one or more first pellets and the one or more second pellets through one or more screens to generate a plurality of isolated microvessels. The enzyme inhibitor of the post-digestion wash may include one or more peptide inhibitors, one or more small molecule inhibitors, one or more native matrix material inhibitors, or combinations thereof.

These and additional features provided by the embodiments of the present disclosure will be more fully understood in view of the following detailed description, in conjunction with the drawings.

### BRIEF DESCRIPTION OF THE SEVERAL VIEWS OF THE DRAWINGS

The following detailed description of specific embodiments of the present disclosure can be best understood when read in conjunction with the following drawings, where like structure is indicated with like reference numerals and in which:

FIG. 1 illustrates a flow chart of a method to isolate microvessels using enriched or purified enzymes to dissociate tissue, according to one or more embodiments as shown and described herein;

FIG. 2 illustrates a flow chart of another method to isolate microvessels using enriched or enzymes to dissociate tissue, according to one or more embodiments as shown and described herein; and

FIG. 3 schematically illustrates a system to implement a computer-based process to automate the methods of the flow charts of FIGS. 1-2, according to one or more embodiments as shown and described herein.

### DETAILED DESCRIPTION

According to the embodiments described herein, systems and a methods are described to isolate intact and functional microvessels using enriched or purified enzymes to dissociate tissue, such as adipose tissue or other tissues. While such enzymes may be described as enriched herein, it is contemplated and within the scope of this disclosure that purified enzymes are a type of enriched enzyme compared to a crude enzyme alone without enrichment or purification. The systems and methods may include use of a double digestion of minced adipose, as described in greater detail below. Additionally or alternatively, the systems and methods may include use of an immediate post-digestion wash, as further described in greater detail below.

In operations in involving crude enzymes, as may be commercially available through WORTHINGTON BIOCHEMICAL of Lakewood, N.J., minced adipose may undergo a single digestion with the crude enzymes and be centrifuged to result in underlying pellets and a disposable top layer. The disposable top layer is discarded, and one or more portions of the underlying pellets may be passed through a screen to disassociate tissue and isolate microvessels. An example of a method including the single digestion to isolate microvessels using crude enzymes to dissociate tissue is set forth in Hoying J B, Boswell C A, Williams S K, Angiogenic Potential of Microvessel Fragments established in Three-Dimensional Collagen Gels, In Vitro Cell Dev. Bio. Anim 1996 July, 32(7): 406-19.

The systems and methods described herein are directed to a double digestion feature in a method to isolate microvessels using enriched or purified enzymes to dissociate tissue, the double digestion feature directed to a second digestion and use of the top layer resulting from a first digestion. In embodiments, the enriched or purified enzymes comprise enriched or purified collagenase. In embodiments, the enriched or purified enzymes comprise chromatographically enriched or purified collagenase. In embodiments, the enriched or purified enzymes comprise a low protease, enriched collagenase product from Clostridium histolyticum useful for isolating cells from tissue. In a specific embodiment, the enriched or purified enzymes comprise Collagenase Gold or DEGold Collagenase or Collagenase HA (as may be commercially available through VITACYTE of Indianapolis, Ind.). In embodiments, in addition to or alternative of Clostridium collagenases, the enriched or purified enzymes may comprise other bacterial sources, such as Vibrio, and/or mammalian collagenases.

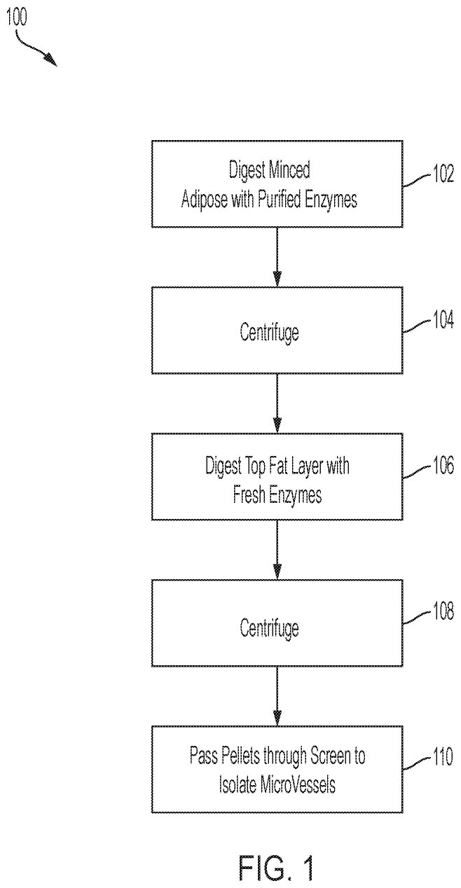

Referring initially to FIG. 1, a flow chart of a method 100 to isolate microvessels using enriched or purified enzymes to dissociate tissue. In block 102, minced adipose undergoes a first digestion with enriched or purified enzymes to result in a first fat-enzyme solution. In embodiments, adipose is minced, such as by hand mixing or via a liposuction cannula and digested with the enriched or purified enzymes. Embodiments of shredding (e.g., mincing) techniques to mince adipose tissue may include use of other mechanical techniques as understood to one of ordinary skill in the art. Such shredding technique may coordinate to receive fat for shredding from one or more procedures such as liposuction (e.g., lipoaspiration) or abdominoplasty. With respect to the liposuction, a surgical cannula may be used to collect fat as lipoaspirates that is then shredded. With respect to abdominoplasty, chunks of fat may be removed and isolated and further shredded.

The first fat-enzyme solution may further include use of another enzyme as a contaminant to assist the enriched or purified enzyme(s) as a catalyst for digestion. The another enzyme may be, but is not limited to, deoxyribonuclease (DNase), which is a nuclease enzyme capable of hydrolyzing phosphodiester bonds that link nucleotides and that, more particularly, catalyze a hydrolytic cleavage of phosphodiester linkages in a DNA backbone to degrade DNA. For a digestion, a digestion flask may be used including a matching volume of fat to a volume of enriched or purified enzyme and a stir bar may be used that may be Teflon or steel coated. In an embodiment, the digestion flask may be shaken or not shaken for 8-10 minutes at 37° C. in a water bath.

In block 104, the first fat-enzyme solution of block 102 is centrifuged in a first centrifuge operation to result in one or more pellets from the first centrifuge operation and a top fat layer disposed above the one or more pellets. The first centrifuge operation allows for a separation of fat and the isolate including microvessels.

In block 106, the top layer resulting from the first centrifuge operation undergoes a second digestion with enriched or purified enzymes to result in a second fat-enzyme solution. In embodiments, the top layer is a top fat layer that is digested in the second digestion with a fresh enriched or purified enzyme to fully release the microvessels from the top fat layer.

In block 108, the second fat-enzyme solution is centrifuged in a second centrifuge operation to result in one or more pellets from the second centrifuge operation. In an embodiment, a lipid/upper layer (e.g., such as an upper lipid layer) and supernatant of the second fat-enzyme solution is aspirated off to leave behind the one or more pellets at a bottom of a tube.

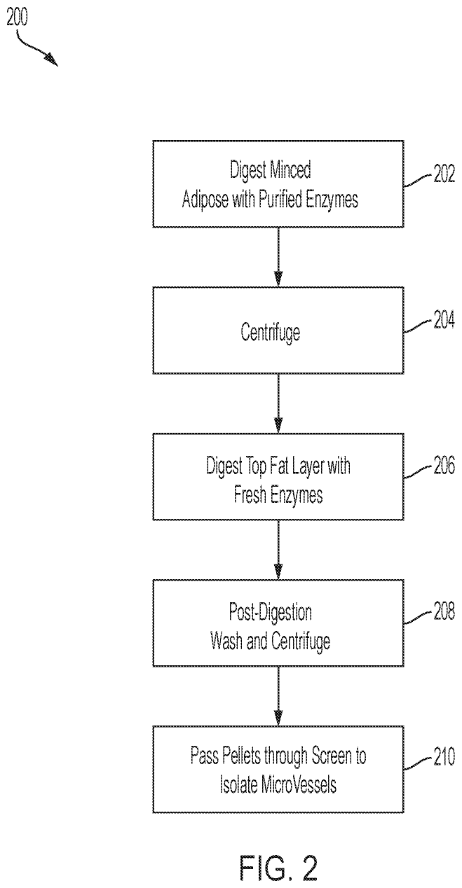

In block 110, portions of the one or more pellets from the first centrifuge operation and from the second centrifuge operation as resulting pellets are passed through one or more screens as isolated microvessels. In an embodiment, the resulting pellets are washed with a gelatin solution and passed through two screens to collect the microvessels. In embodiments, microvessel enzymes may be inhibited and/or quenched to prevent or minimize collagen degradation of the microvessels via cryopreservation and/or a post-digestion wash with an enzyme inhibitor, as described in greater detail below with respect to FIG. 2. The cryopreservation may preserve the microvessels for up to a time period, such as a time period of a month.

### Example 1

In an example procedure, 20 milliliters (mls) of minced adipose tissue is used. A first fat-enzyme solution is prepared, in block 102, of 30 mls of 30 mg Gold Collagenase as the enriched or purified enzyme with 30 mg DNase in 0.1% DPBS as a balanced salt solution to handle and culture mammalian cells. Next, 10 mls of the first fat-enzyme solution is added to 10 mls of fat (e.g., the minced adipose) in a first flask, such that 15 mg of the enriched or purified enzyme is used (e.g., 1.0× enzyme mix). In a second flask, 7.5 mls of the first fat-enzyme solution is added to the other 10 mls of fat such that 7.5 mg of the enriched or purified enzyme is used (e.g., 0.5× enzyme mix). In block 102, both flasks are digested one minute of shaking and 7 minutes without shaking at 37° C. in a water bath for 8 minutes. Both flasks still retain tissue chunks.

In a following step of block 104, the material of both flasks are centrifuged in the first centrifuge operation. The 1.0× enzyme mix results in a larger pellet than the 0.5× enzyme mix. Fat tissue remains at a top layer for each centrifuged mix. In block 106, the top layer is transferred to a new flask and the remaining enzyme of the first fat-enzyme solution is added in at 7.5 mls and redigested. This redigestion acts as a second digestion feature and includes 8 minutes of shaking, resulting in quality tissue with mostly adipocytes at top. After block 104, the 1.0× enzyme mix results in cell clusters with no microvessels in the pellet and the 0.5× enzyme mix results in larger cell clusters with no microvessels in the pellet. After block 106, post the second digestion, and after the centrifuging of block 108 and microvessel isolation of block 110, a considerable number of microvessels result, many of which are long. At 45 count divided by 0.02 ml and multiplied by 40 ml, a result of 90,0000 microvessels is estimated. The resulting microvessels are pelleted and frozen (e.g., cryopreserved) in freeze media 1 ml aliquots at −20° C. for 2-3 hours, then −80° C. if in foam container or directly into −80° C. with a cooling of −1° C./minute. In another embodiment, the 1.0× enzyme mix may be digested in block 102 for 16 minutes.

FIG. 2 illustrates a flow chart of another method 200 to isolate microvessels using enriched or purified enzymes to dissociate tissue. The method 200 is similar to the method 100 of FIG. 1 with an additional option in step 208 to include use of a post-digestion wash, which occurs before the second centrifuge. Thus, the blocks 202, 204, 206, and 210 of FIG. 2 are similar to blocks 102, 104, 106, and 110 as described above. With respect to new block 208, the post-digestion wash may be a feature immediate to the digestion that includes use of a 0.01% porcine gelatin. In an embodiment, the post-digestion wash may include use of an enzyme inhibitor. The enzyme inhibitor may comprise classes of inhibitors such as, but not limited to, peptide inhibitors, small molecule inhibitors, native matrix material inhibitors, or combinations thereof. The native matrix material inhibitors may include, but not be limited to, collagen gels, fibrin, elastin, gelatin, or combinations thereof.

### Example 2

The method of Example 2 starts with sterile supplies and follows best biosafety level 2 (BSL-2) laboratory practices and aseptic technique. The supplies may include the following:
20 μm (˜ϕ60 mm) nylon mesh filter cut round to fit within the bottom of a 100 mm Petri dish.
500 μm (˜ϕ100 mm) nylon mesh filter cut to 10 cm square to be larger than the bottom of a 100 mm Petri dish.
Wire stainless steel mesh screen
125 ml polycarbonate Erlenmeyer flasks with one small stir bar per flask
100 mm×20 mm Petri dish
50 ml conical centrifuge tubes
Steri-flip filter (FISHER SCIENTIFIC Cat # SCGP00525)
200 μl large-orifice micropipette tips
1000 μl pipette tips
Serological pipettes—25, 10 & 5 ml
Benchtop centrifuge
Shaking water bath
0.1% bovine serum albumin protein (BSA) in cation free-phosphate-buffered saline (PBS) as a blocking buffer (BSA-PBS; BSA FISHER SCIENTIFIC Cat # BP1605-100)
Collagenase Gold (VITACYTE Cat #011-1060)
DNase—deoxyribonuclease 1 from bovine pancreas (Sigma DN25)
2% gelatin solution in PBS, autoclaved
Freezing medium (FISHER SCIENTIFIC Cat #12648010)
Hanks Balanced Salt Solution (HBSS; FISHER SCIENTIFIC Cat #14175103)

The procedure of Example 2 with reference to FIG. 2 follows:

The 20 μm nylon screen is placed in a petri dish containing 20 ml of BSA-PBS for later use. For block 202, no more than 35 ml of fat is transferred to a 50 ml conical tube. Up to 4 conical tubes of fat can be used at one time. The volume of each tube is brought to 50 ml with HBSS. The tubes are inverted to wash, and centrifuged at 400 g for 4 min to result in fat at the top of the conical tube as a top fat layer. The top fat layer is moved to fresh 50 ml conical tubes (up to 35 ml per new tube), and the volume is brought up to 50 ml with HBSS, and the fresh tubes are centrifuged at 400 g for 4 min. No more than 30 ml of fat is placed per 125 mL Erlenmeyer flask(s) containing a small spin bar. A first fat-enzyme solution is prepared through a measurement and preparation of Collagenase Gold and DNase solution. The first fat-enzyme solution is made containing 1 mg/ml of each enzyme in BSA-PBS (i.e., 15 mg collagenase and 15 mg DNase in 15 ml BSA-PBS). A volume equal to the volume of fat to be digested is prepared, plus 0.5×ml extra. A 0.2 μm SteriFlip filter is used to sterilize the first fat-enzyme solution.

Next, 1 ml of enzyme solution is added per ml of fat in the flask (i.e., 15 mls of enzyme is added to 15 ml of washed fat). The cap of the flask is tightened and wrapped with parafilm. For the first digestion of block 202, the flask is moved to a 37° C. water bath and shaken for 1 minute, and then incubation is continued in water bath for 7 more minute without shaking.

For the first centrifuge operation of block 204, the first fat-enzyme solution is transferred to 50 ml conical tubes (no more than 35 ml per tube), and the volume of each tube is brought to 50 ml with BSA-PBS. Next, 250 μl of sterile 2% gelatin is added to each conical tube (for a final concentration of 0.01% gelatin) and the first centrifuge operation occurs at 400 g for 4 minutes. Following centrifugation, a pellet results in the conical tube containing blood cells, clumps of stromal cells, and microvessels.

In block 206, the fat in the conical tube is collected at the top (where the fat will appear undigested as a top fat layer) and transferred to the same 50 ml Erlenmeyer flask with stir bar. The same volume of fresh enzyme mix as a second fat-enzyme solution is added as with the first digestion (i.e., 15 mls if started with 15 ml of fat). The cap of the flask is wrapped in parafilm, and the flask is incubated with shaking in a 37° C. water bath for 8 minute in a second digestion. While the fat is being digested in the second digestion, the 50 ml conical tubes may be taken to aspirate the remaining liquid on top of each pellet to leave approximately 5 ml above the pellet. The pellet may be re-suspended and transferred to a petri dish.

In block 208, the resulting fat-enzyme solution may be transferred to a 50 ml conical tube and topped off to 50 ml with BSA-PBS. Further, 250 μl of 2% gelatin may be added to each conical tube (for a final concentration of 0.01% gelatin) and centrifuged in a second centrifuge operation at 400 g for 4 minutes. The resulting pellet will contain the isolated microvessels and un-digestable matrix elements. The lipid/upper layer and supernatant may be aspirated off to leave approximately 5 ml above the pellet disposed at the bottom of the tube.

In block 210, each pellet is re-suspended and 25-30 ml of BSA-PBS is added. The pellets are added to the petri dish including the microvessels (e.g., pellets) from the first digestion. With sterile forceps, the metal screen is placed on top of a new petri dish, and the 500 μm nylon mesh filter is placed on top of the metal screen. The microvessel suspension is slowly pipetted from the dish onto the 500 μm screen to remove pieces of undigested tissue from the suspension. When finished, the petri is rinsed with 10 ml BSA-PBS and pipetted onto the 500 μm screen. The screen is rinsed by pipetting an additional 20 ml of BSA-PBS onto the screen to wash any microvessels through the screen that are adhered to the screen or any tissue chunks. The 500 μm screen is discarded, and the wire support is moved to a new 10 cm dish. At this stage, the microvessels have passed through the screen and are in the dish.

With sterile forceps, the 20 μm nylon mesh filter is placed on top of the wire mesh screen and centered over the new underlying dish. The filtered microvessel suspension, now in the petri dish used for the 500 μm screening, is pipetted through the 20 μm screen in, for example, concentric circles. If the suspension begins to move very slowly through the screen such that a puddle is forming on the screen and taking time to move through, stop this step and proceed to the next step. This slow movement and puddle indicates a high yield of microvessels such that all the single cells may not be able to be washed out if the step is continued. Instead, retrieve a second 20 μm screen, soak briefly in BSA-PBS, and continue with pipetting step using as many additional screens as is necessary. The original dish is rinsed with 10 ml of BSA-PBS to remove any remaining microvessels, and pipette onto the screen.

The screen is rinsed again by gently pipetting 20 ml BSA-PBS in concentric circles to wash out any remaining single cells, taking care not to spill over the edge of the screen. At this stage, the microvessels are trapped on top of the screen with single cells having passed through the screen.

With sterile forceps, the 20 μm nylon mesh filter is slid off the wire mesh and into the petri dish containing 20 mL BSA-PBS that was originally used to soak the screen, microvessel-side up. The 20 μm mesh is allowed to soak for 10 minutes. The petri dish is gently shaken back and forth to dislodge microvessels from the nylon mesh filter. The top of the nylon screen is washed by pipetting up some of the microvessel suspension around the screen, and then pipetted down onto the screen. This suspension is transferred to a 50 ml tube. The wash is repeated several times by pipetting 10 ml at a time of fresh BSA-PBS onto the screen and then moving the rinse to the 50 ml tube. The edges of the screen well should be rinsed as well. The screen should be checked under the microscope to ensure all microvessels have been removed and these removal steps repeated if necessary until all the microvessels have been removed from the screen. The resulting, isolated microvessels should at this stage be in the 50 ml conical tube.

The resulting, isolated microvessels may now be counted. A counting procedure may include first determining that the 50 ml conical tube top is securely tightened. Next, the tube containing the fragment suspension is gently inverted 2-3 times to keep the microvessels distributed evenly in the solution. Two 20 μL samples of the suspension are removed immediately after tube inversions and streaked across a glass slide. The pipette tips are changed between each sample as glass slides are not sterile. Further, it should be determined that no large drops are on the pipette tip before streaking on the slide. Under a microscope, the microvessels are counted in each streak. Microvessels that are stripped (no longer have cells attached) should not be counted. Each branch of large vessels should be counted as a separate vessel. For small capillaries, only every third capillary should be counted.

Next, the total number of microvessels may be calculated through the following Equation 1:

Total Microvessels=(average fragment count from the two 20 μl samples)*[(volume of suspension in the 50 ml tube)/0.02)] (EQUATION 1)

The fragment suspension may be centrifuged at 400 g for 4 minutes, which may be done while counting the microvessels. The supernatant may be decanted or aspirated to leave approximately 100 μL of solution above the microvessel pellet. The 100 μL of solution may be gently pipetted to loosen the microvessel fragments.

The resulting, isolated microvessels may be frozen as well. The cryo-tubes may be prepared such at that all tubes are labelled with their contents (human microvessels (MV), stromal vascular fraction (SVF), etc.), the lot number, number of microvessels in the vial, and preparer initials. The freezing medium is brought to room temperature, and the microvessels are suspended at desired amounts per ml in freezing medium. If freezing medium is not available, DULBECCO MODIFIED EAGLE MEDIUM (DMEM) cell culture media with 20% fetal bovine serum (FBS) and 10% Dimethylsulfoxide (DMSO) may be used. Up to 1 ml of microvessel-freezing medium suspension is transferred to each cryo-tube, while making sure the lid to the tubes are tight, and the microvessels are re-suspended between every cryo-tube. The cryo-tubes are placed into a freezing container (e.g., MR. FROSTY as commercially available from THERMO FISHER SCIENTIFIC) and moved immediately to a −80° C. freezer. If no freezing container is available, a foam shipping container can be used instead. If a foam shipping container is used, the cry-tubes may be placed in −20° C. for 2-4 hours and then in a −80° C. freezer or other freezing device as per instructions. After 24 hours, the vials are transferred to liquid N₂ for storage for retainability.

### Other Examples

The enriched or purified enzyme methods described herein provide for a development of novel isolation, culture, and aliquoting standards to isolate human (or other) microvessels using enriched or purified enzymes to dissociate tissue. Other embodiments are within the scope of this disclosure. By way of example, and not as a limitation, in an embodiment including a single digestion of 8 minutes using enriched or purified enzymes as described herein, a yield is not as a high as when using a double digestion. Further, in an embodiment including a varied enriched or purified enzyme concentration for 8 minutes, a yield is not as a high as when using a double digestion and a fair amount of fat remains undigested. In an embodiment utilizing a double digestion of two rounds at 8 minutes each, a high yield results after the second digestion with a good microvessel quality. Repeating such a double digestion multiple times results in consistent yields and good quality across different fat sources, such as three different fat sources.

When using a double digestion method as described herein of FIG. 1 and prior to freezing, the collagen may quickly degrade with microvessel collapse, likely due to residual collagenase carryover on the microvessel isolate. However, microvessels frozen for more than or equal to four weeks did not result in degraded collagen. To inhibit or inactivate residual collagenase and reduce production time for use of microvessels prior to four weeks, the post-solution wash of block 208 immediate to the second digestion of block 206 and prior the second centrifuge operation maintains collagen integrity throughout culture duration, results in high microvessel yields, and prevents collagen degradation.

Other wash protocols are tested as well in various other examples. Addition of a cysteine (non-comp inhibitor) wash after screening still results in collagen quickly degraded on a first day with a microvessel collapse. Testing of different concentrations of enzyme results in low microvessel yields with lower concentrations but still results in collagen quickly degraded on a first day with a microvessel collapse. An extra PBS wash after an enzyme digestion to rinse out extra enzyme still results in collagen quickly degraded on a first day with a microvessel collapse. A wash with dilute collagen after screening to attempt to competitively bind with remaining enzyme results in collagen polymerization during wash and suspension of collagen chunks with the microvessels, which disrupts the overall structure of the collagen plug. A 0.01% gelatin wash after screening for 5 minutes slows collagen degradation but does not stop it and results in lower quality microvessel growth. A 0.01% gelatin added to all screens/washes after digestions diminishes yield due to gelatin-dependent clumping of microvessels that are screened out, though the collagen did not degrade. A 0.01% gelatin wash after screen for 20 minutes maintains collagen integrity throughout culture duration. A wash after the enzyme digestion with 0.1% gelatin for one wash only results in high yields, good microvessel quality, and collagen that does not degrade.

In embodiments utilizing fetal bovine serum (FBS) containing media, the microvessel growth may be lot dependent and involve expensive and time consuming lot testing. Use of a serum-free medium (SFM) allows for a defined, cost effective process that is not lot dependent and does not include animal products, which is useful for human studies. Use of Sato SFM and 10 ng/ml VEGF results in no microvessel growth. Use of a corrected dosing calculation in a recipe for components in addition to the Sato SFM and 10 ng/ml VEGF results in sluggish microvessel growth. Use of Sato SFM and 50 ng/ml VEGF results in good microvessel growth with a portion of tested lots and sluggish microvessel growth in others, which may be due to gelatin incubation iterations. Use of a media based on human plasma constituents results in good microvessel growth similar to use of Sato SFM and 50 ng/ml VEGF but is an involved and time-consuming medium to make.

In the tested embodiments, the tests were performed on 10K microvessel aliquots at 60,000 microvessels/mL, which 10K microvessel aliquots are customer sized. However, human microvessels are smaller than tested rat microvessels, resulting in a lower density for a same number of microvessels/mL, where a high density is desired for robust microvessel growth. A higher microvessel number per aliquot allows for an easier uniform suspension at thawing and more accurate counts, and a minimum aliquot size of 20K microvessels may provide for easier use and consistent handling. A minimum density of 80K microvessel/mL may be used.

In tested embodiments, some batches of fat contain muscle cells (e.g., myocytes) that can rupture during culture to result in microvessel death. A Percoll centrifugation may separate out cells of different sizes, though microvessels of similar density to myocytes will not separate. Myocytes may stick to plastic, so only the top three-quarters of the isolate may be screened and the myocytes captured by plastic, resulting in a substantially lower myocyte count, good microvessel growth, and a slightly lower microvessel yield. In embodiments, a tissue culture adherent plastic may be utilized to further encourage myocyte adherence.

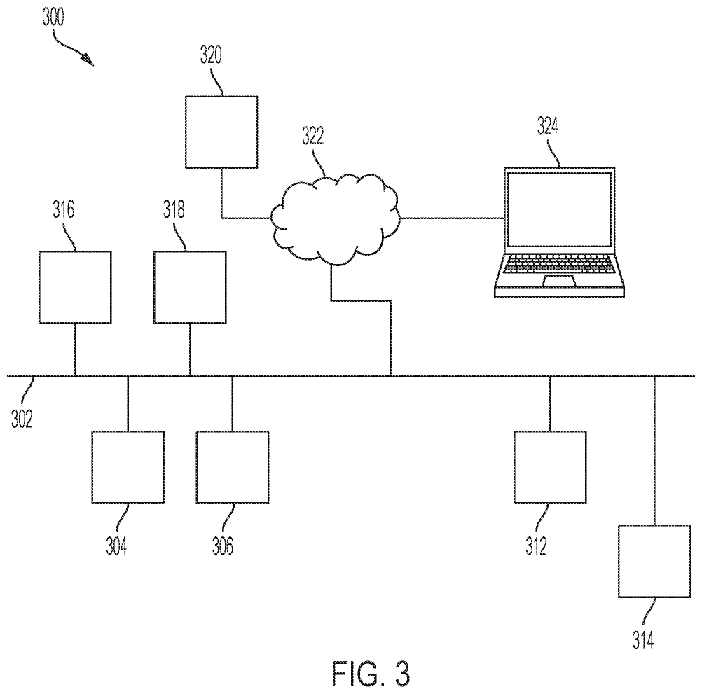

FIG. 3 illustrates a system 300 to implement computer-based control schemes to automate the methods 100, 200 of the flow charts of FIGS. 1-2. The system 300 may be implemented along with using a graphical user interface (GUI) 202 that is accessible at a user workstation (e.g., a computing device 324), for example. The computing device 324 may be a smart mobile device, which may be a smartphone, a tablet, or a like portable handheld smart device. The computing device 324 includes a processor, a memory communicatively coupled to the processor, and machine readable instructions stored in the memory. The machine readable instructions may cause the system 300 to, when executed by the processor, follow one or more of the blocks of the methods 100, 200 of FIGS. 1-2 such that one or more steps of the methods 100, 200 may be automated. Thus, the machine readable instructions may cause the system 300 to, when executed by the processor, follow one or more control schemes as set forth in the one or more processes described herein.

The system 300 includes a communication path 302, one or more processors 304, a memory component 306, a software tool component 312, a storage or database 314 that may include one or more protocols as described herein, an artificial intelligence component 316, a network interface hardware 318, a server 320, a network 322, and at least one computing device 324. The various components of the system 300 and the interaction thereof will be described in detail below.

In some embodiments, the system 300 is implemented using a wide area network (WAN) or network 322, such as an intranet or the Internet, or other wired or wireless communication network that may include a cloud computing-based network configuration. The workstation computing device 324 may include digital systems and other devices permitting connection to and navigation of the network, such as the smart mobile device 200. Other system 300 variations allowing for communication between various geographically diverse components are possible. The lines depicted in FIG. 3 indicate communication rather than physical connections between the various components.

As noted above, the system 300 includes the communication path 302. The communication path 302 may be formed from any medium that is capable of transmitting a signal such as, for example, conductive wires, conductive traces, optical waveguides, or the like, or from a combination of mediums capable of transmitting signals. The communication path 302 communicatively couples the various components of the system 300. As used herein, the term “communicatively coupled” means that coupled components are capable of exchanging data signals with one another such as, for example, electrical signals via conductive medium, electromagnetic signals via air, optical signals via optical waveguides, and the like.

As noted above, the system 300 includes the processor 304. The processor 304 can be any device capable of executing machine readable instructions. Accordingly, the processor 304 may be a controller, an integrated circuit, a microchip, a computer, or any other computing device. The processor 304 is communicatively coupled to the other components of the system 300 by the communication path 302. Accordingly, the communication path 302 may communicatively couple any number of processors with one another, and allow the modules coupled to the communication path 302 to operate in a distributed computing environment. Specifically, each of the modules can operate as a node that may send and/or receive data. The processor 304 may process the input signals received from the system modules and/or extract information from such signals.

As noted above, the system 300 includes the memory component 306 which is coupled to the communication path 302 and communicatively coupled to the processor 304. The memory component 306 may be a non-transitory computer readable medium or non-transitory computer readable memory and may be configured as a nonvolatile computer readable medium. The memory component 306 may comprise RAM, ROM, flash memories, hard drives, or any device capable of storing machine readable instructions such that the machine readable instructions can be accessed and executed by the processor 304. The machine readable instructions may comprise logic or algorithm(s) written in any programming language such as, for example, machine language that may be directly executed by the processor, or assembly language, object-oriented programming (OOP), scripting languages, microcode, etc., that may be compiled or assembled into machine readable instructions and stored on the memory component 306. Alternatively, the machine readable instructions may be written in a hardware description language (HDL), such as logic implemented via either a field-programmable gate array (FPGA) configuration or an application-specific integrated circuit (ASIC), or their equivalents. Accordingly, the methods described herein may be implemented in any conventional computer programming language, as pre-programmed hardware elements, or as a combination of hardware and software components. In embodiments, the system 300 may include the processor 304 communicatively coupled to the memory component 306 that stores instructions that, when executed by the processor 304, cause the processor to perform one or more functions as described herein.

Still referring to FIG. 3, as noted above, the system 300 comprises the display such as a GUI on a screen of the computing device 324 for providing visual output such as, for example, information, graphical reports, messages, or a combination thereof. The computing device 324 may include one or more computing devices across platforms, or may be communicatively coupled to devices across platforms, such as smart mobile devices 200 including smartphones, tablets, laptops, and/or the like. The display on the screen of the computing device 324 is coupled to the communication path 302 and communicatively coupled to the processor 304. Accordingly, the communication path 302 communicatively couples the display to other modules of the system 300. The display can include any medium capable of transmitting an optical output such as, for example, a cathode ray tube, light emitting diodes, a liquid crystal display, a plasma display, or the like. Additionally, it is noted that the display or the computing device 324 can include at least one of the processor 304 and the memory component 306. While the system 300 is illustrated as a single, integrated system in FIG. 3, in other embodiments, the systems can be independent systems.

The system 300 comprises the software tool component 312 to automate steps of one or more protocols or methods as described herein and the artificial intelligence component 316 to train and provide machine learning capabilities to a neural network that may provide machine learning to automatically improve upon and modify automated protocols or methods applied as described herein to result in more accurate and consistent and higher yielding microvessel growth and isolation. In an embodiment, machine readable instructions cause the system 300 to perform at least the following when executed by the one or more processors 304: apply the artificial intelligence component 316 to train a neural network model used by the system 300 to automate one or more protocols of the system 300 as described herein, and apply machine learning to the neural network model via the artificial intelligence component 316 to modify the one or more protocols over time based on historical data associated with the one or more protocols of the system 300 to result in higher yielding microvessel growth and isolation of increasing accuracy and consistency by the system 300 over time. The software tool component 312 and the artificial intelligence component 316 are coupled to the communication path 302 and communicatively coupled to the processor 304. The processor 304 may process the input signals received from the system modules and/or extract information from such signals.

Data stored and manipulated in the system 300 as described herein is utilized by the artificial intelligence component 316, which is able to leverage a cloud computing-based network configuration such as the cloud to apply Machine Learning and Artificial Intelligence. This machine learning application may create models that can be applied by the system 300, to make it more efficient and intelligent in execution. As an example and not a limitation, the artificial intelligence component 316 may include components selected from the group consisting of an artificial intelligence engine, Bayesian inference engine, and a decision-making engine, and may have an adaptive learning engine further comprising a deep neural network learning engine.

The system 300 includes the network interface hardware 318 for communicatively coupling the system 300 with a computer network such as network 322. The network interface hardware 318 is coupled to the communication path 302 such that the communication path 302 communicatively couples the network interface hardware 218 to other modules of the system 300. The network interface hardware 318 can be any device capable of transmitting and/or receiving data via a wireless network. Accordingly, the network interface hardware 318 can include a communication transceiver for sending and/or receiving data according to any wireless communication standard. For example, the network interface hardware 318 can include a chipset (e.g., antenna, processors, machine readable instructions, etc.) to communicate over wired and/or wireless computer networks such as, for example, wireless fidelity (Wi-Fi), WiMax, Bluetooth, IrDA, Wireless USB, Z-Wave, ZigBee, or the like.

Still referring to FIG. 3, data from various applications running on the computing device 324 can be provided from the computing device 324 to the system 300 via the network interface hardware 318. The computing device 324 can be any device having hardware (e.g., chipsets, processors, memory, etc.) for communicatively coupling with the network interface hardware 318 and a network 322. Specifically, the computing device 324 can include an input device having an antenna for communicating over one or more of the wireless computer networks described above.

The network 322 can include any wired and/or wireless network such as, for example, wide area networks, metropolitan area networks, the Internet, an Intranet, the cloud 323, satellite networks, or the like. Accordingly, the network 322 can be utilized as a wireless access point by the computing device 324 to access one or more servers (e.g., a server 320). The server 320 and any additional servers such as a cloud server generally include processors, memory, and chipset for delivering resources via the network 322. Resources can include providing, for example, processing, storage, software, and information from the server 320 to the system 300 via the network 322. Additionally, it is noted that the server 320 and any additional servers can share resources with one another over the network 322 such as, for example, via the wired portion of the network, the wireless portion of the network, or combinations thereof.

The systems and methods utilizing enriched or purified enzymes and a double digestion feature as described herein result in a highly defined isolated microvessel composition, lot-to-lot consistency, and cost-efficiency in comparison to previous methods utilizing crude enzymes as described above.

For the purposes of describing and defining the present disclosure, it is noted that reference herein to a variable being a “function” of a parameter or another variable is not intended to denote that the variable is exclusively a function of the listed parameter or variable. Rather, reference herein to a variable that is a “function” of a listed parameter is intended to be open ended such that the variable may be a function of a single parameter or a plurality of parameters.

It is also noted that recitations herein of “at least one” component, element, etc., should not be used to create an inference that the alternative use of the articles “a” or “an” should be limited to a single component, element, etc.

It is noted that recitations herein of a component of the present disclosure being “configured” or “programmed” in a particular way, to embody a particular property, or to function in a particular manner, are structural recitations, as opposed to recitations of intended use. More specifically, the references herein to the manner in which a component is “configured” or “programmed” denotes an existing physical condition of the component and, as such, is to be taken as a definite recitation of the structural characteristics of the component.

It is noted that terms like “preferably,” “commonly,” and “typically,” when utilized herein, are not utilized to limit the scope of the claimed disclosure or to imply that certain features are critical, essential, or even important to the structure or function of the claimed disclosure. Rather, these terms are merely intended to identify particular aspects of an embodiment of the present disclosure or to emphasize alternative or additional features that may or may not be utilized in a particular embodiment of the present disclosure.

For the purposes of describing and defining the present disclosure it is noted that the terms “substantially” and “approximately” are utilized herein to represent the inherent degree of uncertainty that may be attributed to any quantitative comparison, value, measurement, or other representation. The terms “substantially” and “approximately” are also utilized herein to represent the degree by which a quantitative representation may vary from a stated reference without resulting in a change in the basic function of the subject matter at issue.

Having described the subject matter of the present disclosure in detail and by reference to specific embodiments thereof, it is noted that the various details disclosed herein should not be taken to imply that these details relate to elements that are essential components of the various embodiments described herein, even in cases where a particular element is illustrated in each of the drawings that accompany the present description. Further, it will be apparent that modifications and variations are possible without departing from the scope of the present disclosure, including, but not limited to, embodiments defined in the appended claims. More specifically, although some aspects of the present disclosure are identified herein as preferred or particularly advantageous, it is contemplated that the present disclosure is not necessarily limited to these aspects.

It is noted that one or more of the following claims utilize the term “wherein” as a transitional phrase. For the purposes of defining the present disclosure, it is noted that this term is introduced in the claims as an open-ended transitional phrase that is used to introduce a recitation of a series of characteristics of the structure and should be interpreted in like manner as the more commonly used open-ended preamble term “comprising.”

What is claimed is:

1. A system to isolate microvessels using enriched enzymes to dissociate tissue, the system comprising:
one or more processors;
a non-transitory memory communicatively coupled to the one or more processors; and
machine readable instructions stored in the non-transitory memory that cause the system to perform at least the following, as one or more protocols, when executed by the one or more processors:
digest, in a first digestion, a minced adipose with an enriched enzyme to generate a first fat-enzyme solution;
centrifuge the first fat-enzyme solution from the first digestion in a first centrifuge operation to generate one or more first pellets and a top fat layer disposed above the one or more first pellets;
digest, in a second digestion, the top fat layer with the enriched enzyme to generate a second fat-enzyme solution;
centrifuge the second fat-enzyme solution from the second digestion in a second centrifuge operation to generate one or more second pellets; and
pass one or more portions of the one or more first pellets and the one or more second pellets through one or more screens to generate a plurality of isolated microvessels.

2. The system of claim 1, wherein the enriched enzyme comprises an enriched or purified enzyme comprising a low protease, enriched collagenase product from Clostridium histolyticum to isolate cells from tissue.

3. The system of claim 1, wherein the machine readable instructions further cause the system to perform at least the following when executed by the one or more processors:
prior to the second centrifuge operation, wash the second fat-enzyme solution with an enzyme inhibitor in a post-digestion wash.

4. The system of claim 3, wherein the enzyme inhibitor of the post-digestion wash comprises one or more peptide inhibitors, one or more small molecule inhibitors, one or more native matrix material inhibitors, or combinations thereof.

5. The system of claim 1, wherein the machine readable instructions further cause the system to perform at least the following when executed by the one or more processors:
wash the one or more portions with a gelatin solution; and
pass the one or more portions through two screens to generate the plurality of isolated microvessels.

6. The system of claim 5, wherein the machine readable instructions further cause the system to perform at least the following when executed by the one or more processors:
cyropreserve the plurality of isolated microvessels.

7. The system of claim 1, wherein the machine readable instructions further cause the system to perform at least the following when executed by the one or more processors:
use an additional enzyme as a catalyst for digestion of the first fat-enzyme solution.

8. The system of claim 7, wherein the additional enzyme comprises deoxyribonuclease (DNase).

9. The system of claim 1, wherein the machine readable instructions further cause the system to perform at least the following when executed by the one or more processors:
aspirate off an upper lipid layer and supernatant of the second fat-enzyme solution from the second centrifuge operation to leave behind the one or more second pellets at a bottom of a tube containing the second fat-enzyme solution.

10. The system of claim 1, wherein the machine readable instructions further cause the system to perform at least the following when executed by the one or more processors:
mince an adipose to generate the minced adipose via use of hand mixing, a liposuction cannula, or combinations thereof.

11. The system of claim 10, wherein the adipose is received from one or more procedures comprising liposuction, abdominoplasty, or combinations thereof.

12. The system of claim 1, wherein the machine readable instructions further cause the system to perform at least the following when executed by the one or more processors:
apply an artificial intelligence component to train a neural network model used by the system to automate the one or more protocols of the system.

13. The system of claim 12, wherein the machine readable instructions further cause the system to perform at least the following when executed by the one or more processors:
apply machine learning to the neural network model via the artificial intelligence component to modify the one or more protocols over time based on historical data associated with the one or more protocols of the system to result in higher yielding microvessel growth and isolation of increasing accuracy and consistency by the system over time.

14. A method to isolate microvessels using enriched enzymes to dissociate tissue, the method comprising:
digesting, in a first digestion, a minced adipose with an enriched enzyme to generate a first fat-enzyme solution;
centrifuging the first fat-enzyme solution from the first digestion in a first centrifuge operation to generate one or more first pellets and a top fat layer disposed above the one or more first pellets;
digesting, in a second digestion, the top fat layer with the enriched enzyme to generate a second fat-enzyme solution;
centrifuging the second fat-enzyme solution from the second digestion in a second centrifuge operation to generate one or more second pellets; and
passing one or more portions of the one or more first pellets and the one or more second pellets through one or more screens to generate a plurality of isolated microvessels.

15. The method of claim 14, further comprising:
prior to the second centrifuge operation, washing the second fat-enzyme solution with an enzyme inhibitor in a post-digestion wash.

16. The method of claim 15, wherein the enzyme inhibitor of the post-digestion wash comprises one or more peptide inhibitors, one or more small molecule inhibitors, one or more native matrix material inhibitors, or combinations thereof.

17. The method of claim 16, wherein the enzyme inhibitor of the post-digestion wash comprises 0.01% porcine gelatin.

18. The method of claim 14, further comprising:
using an additional enzyme as a catalyst for digestion of the first fat-enzyme solution.

19. A method to isolate microvessels using enriched enzymes to dissociate tissue, the method comprising:
digesting, in a first digestion, a minced adipose with an enriched enzyme to generate a first fat-enzyme solution;
using an additional enzyme as a catalyst for digestion of the first fat-enzyme solution;
centrifuging the first fat-enzyme solution from the first digestion in a first centrifuge operation to generate one or more first pellets and a top fat layer disposed above the one or more first pellets;
digesting, in a second digestion, the top fat layer with the enriched enzyme to generate a second fat-enzyme solution;
washing the second fat-enzyme solution with an enzyme inhibitor in a post-digestion wash, wherein the enzyme inhibitor of the post-digestion wash comprises one or more peptide inhibitors, one or more small molecule inhibitors, one or more native matrix material inhibitors, or combinations thereof;
centrifuging the second fat-enzyme solution from the second digestion in a second centrifuge operation to generate one or more second pellets; and
passing one or more portions of the one or more first pellets and the one or more second pellets through one or more screens to generate a plurality of isolated microvessels.

20. The method of claim 19, wherein the enzyme inhibitor of the post-digestion wash comprises 0.01% porcine gelatin.
